(19) United States
(12) Patent Application Publication
(10) Pub. No.: US 2023/0120970 A1
(43) Pub. Date: Apr. 20, 2023

(54) SHOP SYSTEM, PROCESSING METHOD, AND NON-TRANSITORY STORAGE MEDIUM

(71) Applicant: NEC Corporation, Minato-ku, Tokyo (JP)

(72) Inventors: Yue CHENG, Tokyo (JP); Mitsunori MORISAKI, Tokyo (JP)

(73) Assignee: NEC Corporation, Minato-ku, Tokyo (JP)

(21) Appl. No.: 17/914,832

(22) PCT Filed: Mar. 24, 2021
(86) PCT No.: PCT/JP2021/012195

§ 371 Date: Sep. 27, 2022

(30) Foreign Application Priority Data
Apr. 10, 2020 (JP) 2020-070797

(51) Int. Cl. G07C 9/00; H04N 7/18; G06T 11/00; G06V 40/16; G06V 40/50

(52) U.S. Cl. CPC G07C 9/00563; G06T 11/00; G06V 40/50; G06V 40/166; G06V 40/171; H04N 7/183

(57) ABSTRACT

A shop system (10) including: an acquisition unit (11) that acquires an entrance image generated by a camera installed at a shop entrance; a detection unit (12) that detects a face of a person in the entrance image; a editing unit (13) that generates a edited image acquired by editing the entrance image in which a face of a person is detected; a display control unit (14) that displays the edited image; a determination unit (15) that determines whether the entrance image prior to editing of the edited image being displayed at a predetermined image capture timing satisfies a predetermined condition; and a processing unit (16) that registers, when the predetermined condition is satisfied, the entrance image determined as satisfying the predetermined condition in shop entry person information stored by a storage unit, and also sets an entry control apparatus installed at a shop entrance into an entry-allowed state.

### TECHNICAL FIELD

The present invention relates to a shop system, a processing method, and a program.

### BACKGROUND ART

Non-Patent Document 1 discloses a system that is a system used in an unmanned shop in which, when an image of a face is captured at an entrance, shop entry is made possible.

Patent Document 1 discloses a technique for analyzing an image captured in such a way as to include a user operating a kiosk terminal and a person present behind the user, generates, when detecting a suspicious person, an image added with information discriminating the user from the suspicious person, and displays the image on an operator terminal used for an operator residing in a call center or the like.

Patent Document 2 discloses a technique for performing, based on face authentication, entry control.

Patent Document 3 discloses a technique for displaying, in preview display, a edited image acquired by editing a captured image, instead of directly displaying the captured image.

### Related Document

### Patent Document

[Patent Document 1] Japanese Patent Application Publication No. 2019-149625
[Patent Document 2] Japanese Patent Application Publication No. 2018-128970
[Patent Document 3] Japanese Patent Application Publication No. 2011-97519

### Non-Patent Document

[Non-Patent Document 1] “Shop entry by taking a face photo...Demonstration experiments of midnight unmanned services conducted by Lawson for countermeasures against shortage of workers” [online], Aug. 23, 2019, FNN PRIME, [searched on Mar. 12, 2020], the Internet<URL: https://www.fnn.jp/posts/00047868HDK/201908232030_livenewsalpha_HDK>

### DISCLOSURE OF THE INVENTION

### Technical Problem

In an unmanned shop and a labor-saving shop, in order to ensure a safe transaction, a technique for determining a person having entered a shop is required. As one example, as in the technique disclosed in Non-Patent Document 1, a technique in which face image capture is a condition for shop entry is conceivable. In this case, a person having entered a shop can be determined based on a face photo of a shop entry person. However, some persons feel resistance in face image capture at a time of shop entry. Therefore, when face image capture is a condition for shop entry, the number of shop users may decrease. Any of Patent Documents 1 to 3 and Non-Patent Document 1 does not disclose the problem.

An issue of the present invention is to reduce a decrease in the number of shop users caused by face image capture as a condition for shop entry

### Solution to Problem

According to the present invention, provided is a shop system including:
an acquisition unit that acquires an entrance image generated by a camera installed at a shop entrance;
a detection unit that detects a face of a person in the entrance image;
a editing unit that generates a edited image acquired by editing the entrance image in which a face of a person is detected;
a display control unit that displays, on a display unit, the edited image;
a determination unit that determines whether the entrance image prior to editing of the edited image being displayed by the display unit at a predetermined image capture timing satisfies a predetermined condition; and
a processing unit that registers, when the predetermined condition is satisfied, the entrance image determined as satisfying the predetermined condition in shop entry person information stored by a storage unit, and also setting an entry control apparatus installed at a shop entrance into an entry-allowed state.

Further, according to the present invention, provided is a processing method including, by a computer:
acquiring an entrance image generated by a camera installed at a shop entrance;
detecting a face of a person in the entrance image;
generating a edited image acquired by editing the entrance image in which a face of a person is detected;
displaying, on a display unit, the edited image;
determining whether the entrance image prior to editing of the edited image being displayed by the display unit at a predetermined image capture timing satisfies a predetermined condition; and
registering, when the predetermined condition is satisfied, the entrance image determined as satisfying the predetermined condition in shop entry person information stored by a storage unit, and also setting an entry control apparatus installed at a shop entrance into an entry-allowed state.

Further, according to the present invention, provided is a program causing a computer to function as:
an acquisition unit that acquires an entrance image generated by a camera installed at a shop entrance;
a detection unit that detects a face of a person in the entrance image;
a editing unit that generates a edited image acquired by editing the entrance image in which a face of a person is detected;
a display control unit that displays, on a display unit, the edited image;
a determination unit that determines whether the entrance image prior to editing of the edited image being displayed by the display unit at a predetermined image capture timing satisfies a predetermined condition; and
a processing unit that registers, when the predetermined condition is satisfied, the entrance image determined as satisfying the predetermined condition in shop entry person information stored by a storage unit, and also setting an entry control apparatus installed at a shop entrance into an entry-allowed state.

### Advantageous Effects of Invention

According to the present invention, a decrease in the number of shop users caused by face image capture as a condition for shop entry can be reduced.

### BRIEF DESCRIPTION OF THE DRAWINGS

FIG. 1 is a diagram for illustrating an outline of a shop system according to the present example embodiment.

FIG. 2 is a diagram illustrating one example of a hardware configuration of the shop system according to the present example embodiment.

FIG. 3 is one example of a function block diagram of the shop system according to the present example embodiment.

FIG. 4 is a diagram for illustrating one example of image editing performed by the shop system according to the present example embodiment.

FIG. 5 is a diagram schematically illustrating one example of information processed by the shop system according to the present example embodiment.

FIG. 6 is a flowchart illustrating one example of a flow of processing of the shop system according to the present example embodiment.

FIG. 7 is a flowchart illustrating one example of a flow of processing of the shop system according to the present example embodiment.

FIG. 8 is a flowchart illustrating one example of a flow of processing of the shop system according to the present example embodiment.

### DESCRIPTION OF EMBODIMENTS

### First Example Embodiment

### Outline

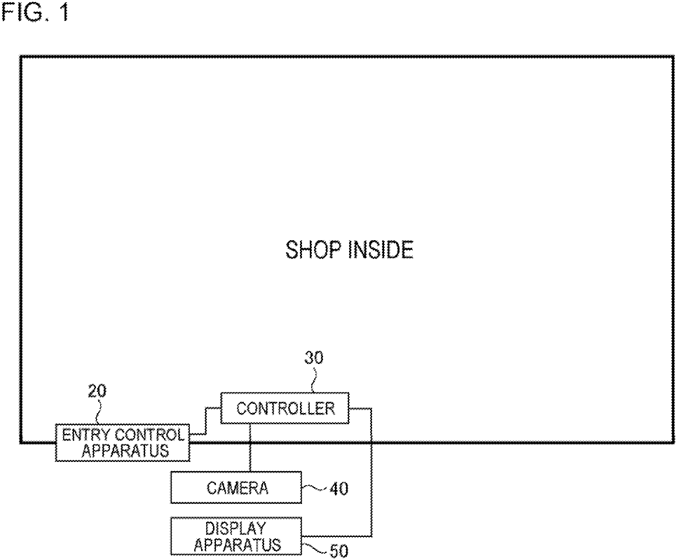

First, by using FIG. 1, an outline of a shop system according to the present example embodiment is described. FIG. 1 illustrates an entry control apparatus 20, a controller 30, a camera 40, and a display apparatus 50. The shop system includes at least the controller 30. The shop system may further include any one of the entry control apparatus 20, the camera 40, and the display apparatus 50. The controller 30 is communicably connected to each of the entry control apparatus 20, the camera 40, and the display apparatus 50 in a wired and/or wireless manner.

The entry control apparatus 20, the camera 40, and the display apparatus 50 are installed at an entrance of a shop. The entry control apparatus 20 is an apparatus that controls entry to a shop, and, for example, a gate, an automatic door, and the like are exemplified. The camera 40 is installed in order to capture an image of a face of a shop visitor. The display apparatus 50 displays an image generated by the camera 40 in such a way as to be browsable by a shop visitor. Note that, “shop entry” indicates that the entry control apparatus 20 is passed and entry is made into a shop. “Shop visit” indicates coming to a shop and does not specifically indicate whether entry is made into a shop.

A shop visitor performs work for capturing, by the camera 40, an image of his/her own face prior to shop entry. Specifically, first, a shop visitor locates a his/her own face in front of the camera 40, browses an image being displayed by the display apparatus 50, and adjusts a location and an expression of the own face. At that time, the controller 30 does not directly display, on the display apparatus 50, an image generated by the camera 40, but displays, on the display apparatus 50, a edited image after editing of the image. In this manner, while image capture work for shop entry is performed, instead of an image itself acquired by capturing an image of a shop visitor, a edited image acquired by editing the image is displayed in a browsable manner, and therefore the shop visitor can perform image capture work for shop entry while enjoying the edited image.

When image capture work for shop entry is normally completed, the controller 30 sets the entry control apparatus 20 into an entry-allowed state. Based on the control, a gate (entry control apparatus 20) is set into an open state or an automatic door (entry control apparatus 20) is set into an open state.

According to such a shop system, a shop visitor can perform image capture work for shop entry while enjoying. Therefore, resistance of a shop visitor to the image capture work can be reduced. As a result, a decrease in the number of shop users caused by face image capture as a condition for shop entry can be reduced.

### Hardware Configuration

Next, one example of a hardware configuration of the shop system is described. A function unit included in the shop system according to the present example embodiment is achieved by any combination of hardware and software mainly including a central processing unit (CPU) of any computer, a memory, a program loaded onto a memory, a storage unit (capable of storing, in addition to a program previously stored from a stage where an apparatus is shipped, a program downloaded from a storage medium such as a compact disc (CD), a server on the Internet, and the like) such as a hard disk storing the program, and a network-connection interface. Then, it should be understood by those of ordinary skill in the art that, in an achievement method and an apparatus for the above, there are various modified examples.

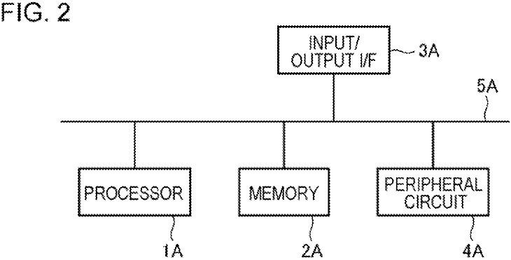

FIG. 2 is a block diagram illustrating a hardware configuration of the shop system according to the present example embodiment. As illustrated in FIG. 2, the shop system includes a processor 1A, a memory 2A, an input/output interface 3A, a peripheral circuit 4A, and a bus 5A. The peripheral circuit 4A includes various modules. Note that, the peripheral circuit 4A may not necessarily be included. Note that, the shop system may be configured by one apparatus united physically and/or logically, or may be configured by a plurality of apparatuses separated physically and/or logically. When a configuration is made by a plurality of apparatuses separated physically and/or logically, each of the plurality of apparatuses can include the hardware configuration described above.

The bus 5A is a data transmission path in which the processor 1A, the memory 2A, the peripheral circuit 4A, and the input/output interface 3A mutually transmit/receive data. The processor 1A is an arithmetic processing unit, for example, such as a CPU and a graphics processing unit (GPU). The memory 2A is a memory, for example, such as a random access memory (RAM) and a read only memory (ROM). The input/output interface 3A includes an interface for acquiring information from an input apparatus, an external apparatus, an external server, an external sensor, the entry control apparatus 20, the camera 40, the display apparatus 50, and the like, an interface for outputting information to an output apparatus, an external apparatus, an external server, the entry control apparatus 20, the camera 40, the display apparatus 50, and the like, and the like. The input apparatus is, for example, a keyboard, a mouse, a microphone, a touch panel, a physical button, a camera, and the like. The output apparatus is, for example, a display, a speaker, a printer, a mailer, and the like. The processor 1A issues an instruction to each module, and thereby, can perform an arithmetic operation, based on arithmetic results of the modules.

### Function Configuration

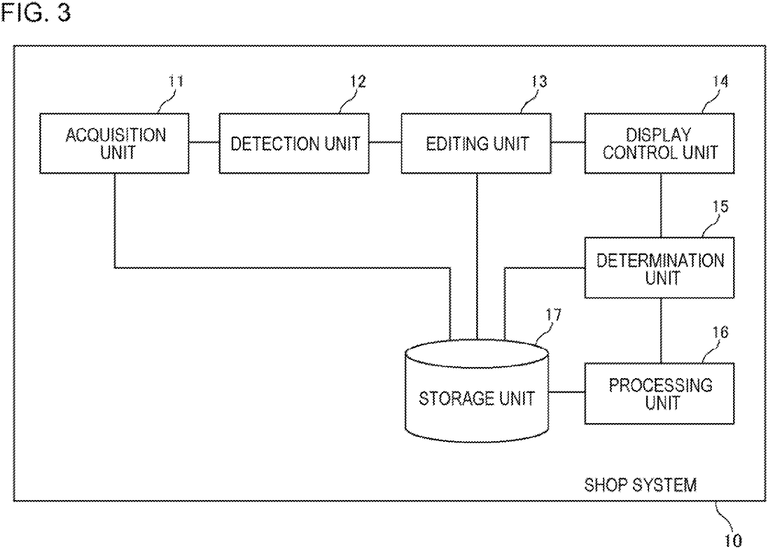

Next, a function configuration of a shop system 10 is described. FIG. 3 illustrates one example of a function block diagram of the shop system 10. As illustrated, the shop system 10 includes an acquisition unit 11, a detection unit 12, a editing unit 13, a display control unit 14, a determination unit 15, a processing unit 16, and a storage unit 17.

The acquisition unit 11 acquires an image (hereinafter, referred to as an “entrance image”) generated by the camera 40 installed at a shop entrance. The acquisition unit 11 acquires, based on real-time processing, an entrance image from the camera 40.

The detection unit 12 detects a face of a person in an entrance image. “A technique for detecting a face of a person in an image” is widely known, and therefore description thereof is omitted herein.

The editing unit 13 generates a edited image acquired by editing an entrance image in which a face of a person is detected. Note that, the editing unit 13 may or may not necessarily execute processing of editing an entrance image in which a face of a person is not detected. However, it is highly possible that an entrance image in which a face of a person is not detected is not browsed by any person. By omitting processing of editing such an entrance image, a processing load on the editing unit 13 can be reduced.

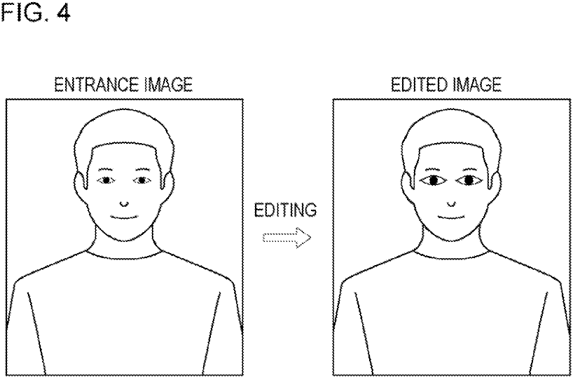

There are various editing contents. For example, as illustrated in FIG. 4, processing of editing a face portion of a person in an image may be executed. Specifically, processing of editing may be executed in such a way that a feature value extracted from a face of a person included in an entrance image and a feature value extracted from a face of a person included in a edited image are not matched with each other. In an illustrated example, based on editing, a size of each of eyes of a person is changed.

In addition, processing of editing another portion (a background portion, a body portion of a person, or the like) in an image may be executed. For example, editing for displaying, in an overlapped manner, a frame, a decoration, and the like on a background portion may be performed, editing for displaying, in an overlapped manner, predetermined clothes (a tuxedo, a dress, or the like) on a body portion of a person may be performed, or another piece of editing may be performed.

The display control unit 14 displayed, on the display apparatus 50 (a display unit), a edited image generated by the editing unit 13. The display apparatus 50 displays a edited image in such a way as to be browsable by a shop visitor (a shop visitor whose image is being captured by the camera 40) being performing image capture work for shop entry in front of the camera 40. The display apparatus 50 may be, for example, a display installed in a location and in a direction browsable by a shop visitor being performing image capture work for shop entry in front of the camera 40. In addition, the display apparatus 50 may be a projection apparatus that projects information in a location browsable by a shop visitor being performing image capture work for shop entry in front of the camera 40.

The determination unit 15 determines whether an entrance image prior to editing of a edited image being displayed by the display apparatus 50 at a predetermined image capture timing satisfies a predetermined condition.

The predetermined image capture timing may be a timing at which a shop visitor inputs an image capture instruction. In this case, a shop visitor inputs an image capture instruction via any input apparatus such as a touch panel, a physical button, a microphone, a mouse, and a keyboard. In addition, the predetermined image capture timing may be a timing at which a predetermined time elapses from a predetermined reference time point. The predetermined reference time may be, for example, a timing of detecting, by using a human sensor, a camera, or the like, that a shop visitor approaches the camera 40, may be a timing of starting display (display based on the display apparatus 50) of a edited image acquired by editing an entrance image including a face of the shop visitor, or may be another timing.

The determination unit 15 determines, when detecting coming of a predetermined image capture timing, a edited image being displayed by the display apparatus 50 at the time pint, and then acquires an entrance image prior to editing of the determined edited image. For example, an entrance image acquired by the acquisition unit 11 and a edited image generated by the editing unit 13 may be temporarily stored in the storage unit 17 in association with each other. Then, the determination unit 15 may acquire, from among entrance images stored in the storage unit 17, an entrance image associated with the determined edited image. In addition, the determination unit 15 performs editing for returning a edited image to an entrance image, and thereby, may acquire an entrance image prior to editing of the determined edited image.

The determination unit 15 acquires an entrance image prior to editing of the determined edited image, and thereafter, determines whether the acquired entrance image satisfies a predetermined condition. The predetermined condition is a fact that “a content of an entrance image is a content capable of determining an individual”, and, for example, a fact that “a predetermined feature value of a face of a person can be extracted”, a fact that “a predetermined part (an eye, a nose, a mouse, or the like) of a face of a person can be extracted”, and the like are exemplified.

The processing unit 16 registers, when the determination unit 15 determines that a predetermined condition is satisfied, an entrance image determined as satisfying the predetermined condition in shop entry person information stored in the storage unit 17, and also sets the entry control apparatus 20 into an entry-allowed state.

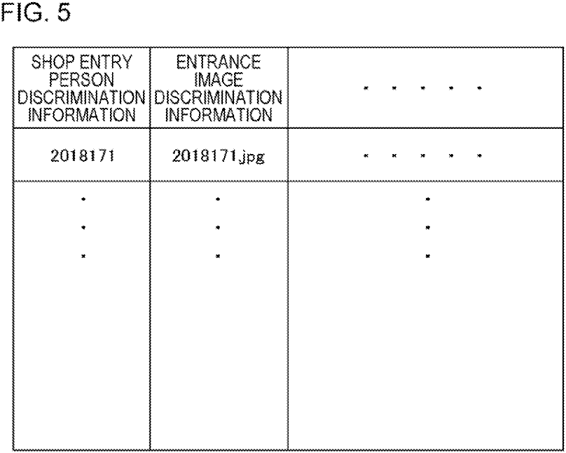

FIG. 5 schematically illustrates one example of shop entry person information. In the illustrated example, a piece of shop entry person discrimination information for discriminating shop entry persons from each other and a piece of information (a file name or the like) discriminating an entrance image determined as satisfying a predetermined condition are registered in association with each other. Based on the shop entry person information, a shop entry person can be determined.

Note that, in a case of a technique for editing an image providing entertainment as a main purpose and displaying the edited image in a browsable manner, a edited image being displayed at an image capture timing is stored but an image prior to editing is not stored. In contrast, the shop system 10 that acquires, as a main purpose, information (face image) for determining a shop entry person and generates and displays a edited image in order to decrease resistance to image capture stores an image prior to editing of a edited image being displayed at an image capture timing. In this point, a technique for editing an image providing entertainment as a main purpose and displaying the edited image in a browsable manner and the shop system 10 are different from each other.

The entry control apparatus 20 is an apparatus that controls entry to a shop, and is, for example, a gate or an automatic door. The entry control apparatus 120, a first state (a state where a gate or a door is open) allowing passing (entry to a shop), and a second state (a state where a gate or a door is closed) rejecting passing (entry to a shop) may be taken. Then, the entry control apparatus 20 performs, based on a control signal (electrical signal) transmitted by the processing unit 16, an operation for transition between the first state and the second state.

The processing unit 16 transmits, when the determination unit 15 determines that a predetermined condition is satisfied, a control signal for being set into the first state to the entry control apparatus 20. The entry control apparatus 20 is set into the first state, based on the control signal.

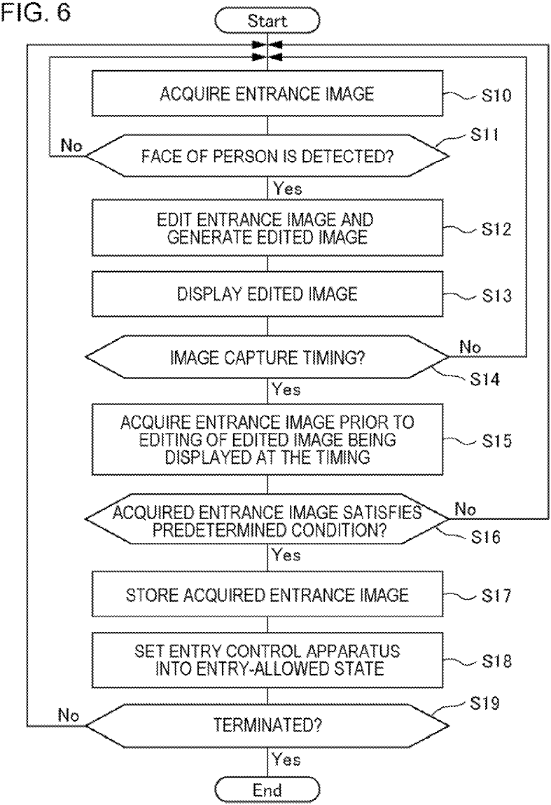

Next, by using a flowchart in FIG. 6, one example of a flow of processing of the shop system10 is described.

First, the acquisition unit 11 acquires an image (entrance image) generated by a camera 40 installed at a shop entrance (S10). The acquisition unit 11 acquires, based on real-time processing, an entrance image from the camera 40. The camera 40 may always execute generation of an image. In addition, the camera 40 may execute generation of an image while, by using a human sensor or the like, it is detected that a person is present in a predetermined location in front of the camera 40.

Thereafter, the detection unit 12 detects a face of a person in an entrance image (S11). When a face of a person is not detected in the entrance image (No in S11), a return is made to S10 and similar processing is repeated.

On the other hand, when a face of a person is detected in the entrance image (Yes in S11), the editing unit 13 edits the entrance image, and generates a edited image (S12). Then, the display control unit 14 displays the generated edited image on the display apparatus 50 (S13).

Note that, the display control unit 14 may display, prior to display of a edited image, on the display apparatus 50, a live image (an image not including a face of a person) acquired by the acquisition unit 11 from the camera 40, may display another piece of information (an advertisement, weather, news, and the like), or may display nothing.

Thereafter, when a predetermined image capture timing does not come (No in S14), a return is made to S10 and similar processing is repeated. As described above, the predetermined image capture timing may be a timing at which a shop visitor inputs an image capture instruction, may be a timing at which a predetermined time elapses from a predetermined reference time point, or may be another timing.

When a predetermined image capture timing comes (Yes in S14), the determination unit 15 determines a edited image being displayed by the display apparatus 50 at that time, and then acquires an entrance image prior to editing of the determined edited image (S15). Then, the determination unit 15 determines whether the acquired entrance image satisfies a predetermined condition. As described above, the predetermined condition is a fact that “a content of an entrance image is a content capable of determining an individual”, and, for example, a fact that “a predetermined feature value of a face of a person can be extracted”, a fact that “a predetermined part (an eye, a nose, a mouse, or the like) of a face of a person can be extracted”, and the like are exemplified.

When the acquired entrance image does not satisfy the predetermined condition (No in S16), a return is made to S10 and similar processing is repeated. Note that, at that time, the shop system 10 may display, on the display apparatus 50, an error message such as a message that “A face image has not been correctly captured. Please, capture an image once again.”.

On the other hand, when the acquired entrance image satisfies the predetermined condition (Yes in S16), the processing unit 16 newly registers the acquired entrance image in shop entry person information stored by the storage unit 17 (S17). Then, the processing unit 16 executes control for setting the entry control apparatus 20 into an entry-allowed state (a state where a gate or a door is open) (S18).

### Advantageous Effect

As described above, the shop system 10 according to the present example embodiment displays, while a shop visitor performs work for capturing an image of a face prior to shop entry, instead of an image itself acquired by capturing an image of a face of a shop visitor, a edited image acquired by editing the image in such a way as to be browsable by the shop visitor. According to such a shop system 10, a shop visitor can perform image capture work for shop entry while enjoying a edited image. Therefore, resistance of a shop visitor to the image capture work can be reduced. As a result, a decrease in the number of shop users caused by face image capture as a condition for shop entry can be reduced.

Further, the shop system 10 can edit an image in such a way that a feature value extracted from a face of a person included in an entrance image and a feature value extracted from a face of a person included in a edited image are not matched with each other. When a face of a person is edited in this manner, browsing of a edited image is more enjoyable. As a result, resistance of a shop visitor to image capture work prior to shop entry can be further reduced.

### Second Example Embodiment

A shop system 10 according to the present example embodiment is different from the first example embodiment in that a function of storing a edited image being displayed at an image capture timing and providing the stored edited image for a customer is included.

A processing unit 16 executes processing of storing, in a storage unit 17, a edited image being displayed by a display apparatus 50 at a predetermined image capture timing and providing the stored edited image for a shop visitor. In other words, the processing unit 16 according to the present example embodiment stores, in the storage unit 17, both of “a edited image being displayed by the display apparatus 50 at a predetermined image capture timing” and “an entrance image prior to editing of the edited image”.

Herein, one example of processing of providing a stored edited image for a shop visitor is described. Note that, exemplification herein is merely one example, and an achievement means is not limited thereto.

### Example 1

The processing unit 16 provides storage location information (a URL or the like) of a edited image for a shop visitor. The processing unit 16 may generate a code (a two-dimensional code or the like) indicating storage location information and display the generated code on the display apparatus 50. In this case, a shop visitor performs an operation for reading the code by operating his/her own user terminal, and thereby, can acquire storage location information. In addition, the processing unit 16 may transmit, according to an operation, by a shop visitor, for holding a user terminal (a smartphone, a smartwatch, a tablet terminal, a mobile phone, or the like) over a predetermined location, storage location information to the user terminal via short-range radio communication. The shop visitor accesses, after acquiring the storage location information, a storage location indicated by the storage location information, by operating his/her user terminal, and acquires a edited image.

### Example 2

The processing unit 16 receives input of customer discrimination information, and provides a edited image for a customer discriminated by the customer discrimination information. For example, customer discrimination information and a mail address may be registered in a center server in association with each other. Then, the processing unit 16 may transmit a edited image or the storage location information to a mail address associated with input customer discrimination information.

In addition, the processing unit 16 may provide a edited image or the storage location information on an application or on an account page (a page after login using customer discrimination information) of a home page.

Note that, the processing unit 16 may receive, from a shop visitor, input of an intention of whether to desire to acquire a edited image at any timing while image capture work for shop entry is performed. Then, when input indicating desire is received, storage of a edited image and provision for a shop visitor are executed, but when input indicating no desire is received, these pieces of processing may not necessarily be executed.

The processing unit 16 deletes, from the storage unit 17 at a predetermined timing, an entrance image and a edited image stored in the storage unit 17. Thereby, protection of privacy and the like are achieved. Note that, due to a difference in purpose between storage of a edited image and storage of an entrance image, timings of deleting each of images are different from each other. Specifically, a timing of deleting a edited image is earlier than a timing of deleting an entrance image.

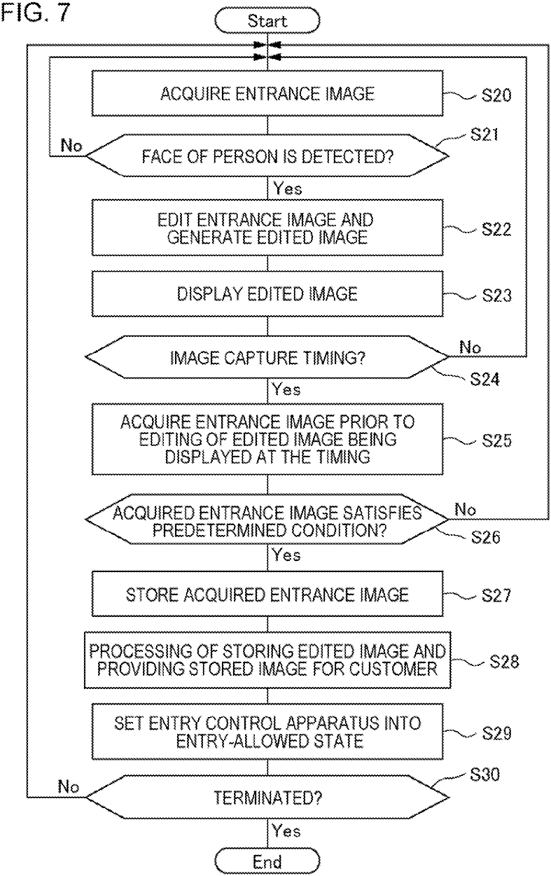

Next, by using a flowchart in FIG. 7, one example of a flow of processing of the shop system 10 is described.

First, an acquisition unit 11 acquires an image (entrance image) generated by a camera 40 installed at a shop entrance (S20). The acquisition unit 11 acquires, based on real-time processing, an entrance image from the camera 40. The camera 40 may always execute generation of an image. In addition, the camera 40 may execute generation of an image while, by using a human sensor or the like, it is detected that a person is present in a predetermined location in front of the camera 40.

Thereafter, a detection unit 12 detects a face of a person in an entrance image (S21). When a face of a person is not detected in the entrance image (No in S21), a return is made to S20 and similar processing is repeated.

On the other hand, when a face of a person is detected in the entrance image (Yes in S21), a editing unit 13 edits the entrance image, and generates a edited image (S22). Then, a display control unit 14 displays the generated edited image on the display apparatus 50 (S23).

Note that, the display control unit 14 may display, prior to display of a edited image, on the display apparatus 50, a live image (an image not including a face of a person) acquired by the acquisition unit 11 from the camera 40, may display another piece of information (an advertisement, weather, news, and the like), or may display nothing.

Thereafter, when a predetermined image capture timing does not come (No in S24), a return is made to S20 and similar processing is repeated. As described above, the predetermined image capture timing may be a timing at which a shop visitor inputs an image capture instruction, may be a timing at which a predetermined time elapses from a predetermined reference time point, or may be another timing.

When a predetermined image capture timing comes (Yes in S24), a determination unit 15 determines a edited image being displayed by the display apparatus 50 at that time, and then acquires an entrance image prior to editing of the determined edited image (S25). Then, the determination unit 15 determines whether the acquired entrance image satisfies a predetermined condition. As described above, the predetermined condition is a fact that “a content of an entrance image is a content capable of determining an individual”, and, for example, a fact that “a predetermined feature value of a face of a person can be extracted”, a fact that “a predetermined part (an eye, a nose, a mouse, or the like) of a face of a person can be extracted”, and the like are exemplified.

When the acquired entrance image does not satisfy the predetermined condition (No in S26), a return is made to S20 and similar processing is repeated. Note that, at that time, the shop system 10 may display, on the display apparatus 50, an error message such as a message that “A face image has not been correctly captured. Please, capture an image once again.”.

On the other hand, when the acquired entrance image satisfies the predetermined condition (Yes in S26), the processing unit 16 newly registers the acquired entrance image in shop entry person information stored by the storage unit 17 (S27). Further, the processing unit 16 executes processing of storing a edited image being displayed by the display apparatus 50 at a predetermined image capture timing and providing the stored edited image for a customer (S28). One example of processing of providing a edited image for a customer is as described above.

Note that, the processing unit 16 may receive, from a shop visitor, input of an intention of whether to desire to acquire a edited image at any timing while image capture work for shop entry is performed. Then, when input indicating desire is received, processing of S28 is executed, but when input indicating no desire is received, processing of S28 may not necessarily be executed.

Thereafter, the processing unit 16 executes control for setting the entry control apparatus 20 into an entry-allowed state (a state where a gate or a door is open) (S29).

Other configurations of the shop system 10 are similar to those of the first example embodiment.

According to the shop system 10 of the present example embodiment, an advantageous effect similar to that of the first example embodiment is achieved. Further, according to the shop system 10 of the present example embodiment, a edited image is stored, and thereby, the stored edited image can be provided for a shop visitor. Thereby, a shop visitor enjoys image capture work prior to shop entry. As a result, resistance to image capture work prior to shop entry can be further reduced.

### Third Example Embodiment

A shop system 10 according to the present example embodiment is different from the first and the second example embodiments in that, based on a face image included in an entrance image, a person attribute is estimated and, by using a method according to an estimation result, an entrance image is edited.

A editing unit 13 estimates a person attribute, based a face of a person detected in an entrance image. For example, the editing unit 13 estimates any attribute such as gender, an age, and nationality, from among attributes capable of being estimated from a face image.

Then, the editing unit 13 edits an entrance image, by using a method according to an estimation result (person attribute). For example, a method of editing is previously determined with respect to each person attribute. As examples of a combination of a person attribute and a method of editing, a matter that “a hair style is formed into a crew cut” when a person attribute includes “a female” and “a teenager”, a matter that “a hair style is formed into a braid” when a person attribute includes “a male” and “a teenager”, a matter that “eyes are made to look bigger” when a person attribute includes “a female” and “a twenty-something and a thirty-something”, and the like are exemplified, but not limited thereto.

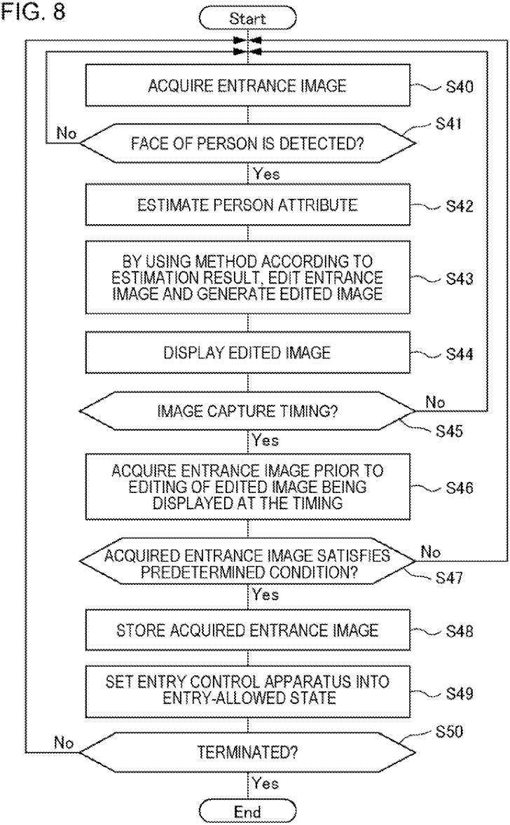

Next, by using a flowchart in FIG. 8, one example of a flow of processing of the shop system 10 is described

First, an acquisition unit 11 acquires an image (entrance image) generated by a camera 40 installed at a shop entrance (S40). The acquisition unit 11 acquires, based on real-time processing, an entrance image from the camera 40. The camera 40 may always execute generation of an image. In addition, the camera 40 may execute generation of an image while, by using a human sensor or the like, it is detected that a person is present in a predetermined location in front of the camera 40.

Thereafter, a detection unit 12 detects a face of a person in an entrance image (S41). When a face of a person is not detected in the entrance image (No in S41), a return is made to S40 and similar processing is repeated.

On the other hand, when a face of a person is detected in the entrance image (Yes in S41), the editing unit 13 estimates, based on the face of the person included in the entrance image, a person attribute of the person (S42). Then, the editing unit 13 edits the entrance image, by using a method according to an estimation result in S42, and generates a edited image (S42). Then, a display control unit 14 displays the generated edited image on a display apparatus 50 (S44).

Note that, the display control unit 14 may display, prior to display of a edited image, on the display apparatus 50, a live image (an image not including a face of a person) acquired by the acquisition unit 11 from the camera 40, may display another piece of information (an advertisement, weather, news, and the like), or may display nothing.

Thereafter, when a predetermined image capture timing does not come (No in S45), a return is made to S40 and similar processing is repeated. As described above, the predetermined image capture timing may be a timing at which a shop visitor inputs an image capture instruction, may be a timing at which a predetermined time elapses from a predetermined reference time point, or may be another timing.

When a predetermined image capture timing comes (Yes in S45), a determination unit 15 determines a edited image being displayed by the display apparatus 50 at that time, and then acquires an entrance image prior to editing of the determined edited image (S46). Then, the determination unit 15 determines whether the acquired entrance image satisfies a predetermined condition. As described above, the predetermined condition is a fact that “a content of an entrance image is a content capable of determining an individual”, and, for example, a fact that “a predetermined feature value of a face of a person can be extracted”, a fact that “a predetermined part (an eye, a nose, a mouse, or the like) of a face of a person can be extracted”, and the like are exemplified.

When the acquired entrance image does not satisfy the predetermined condition (No in S47), a return is made to S40 and similar processing is repeated. Note that, at that time, the shop system 10 may display, on the display apparatus 50, an error message such as a message that “A face image has not been correctly captured. Please, capture an image once again.”.

On the other hand, when the acquired entrance image satisfies the predetermined condition (Yes in S47), a processing unit 16 newly registers the acquired entrance image in shop entry person information stored by a storage unit 17 (S48). Then, the processing unit 16 executes control for setting an entry control apparatus 20 into an entry-allowed state (a state where a gate or a door is open) (S49).

Note that, after S48, “processing (S28 in FIG. 7) for storing a edited image being displayed by the display apparatus 50 at an image capture timing and providing the stored edited image for a shop visitor” described according to the second example embodiment may be executed.

Herein, a modified example according to the present example embodiment is described.

The display apparatus 50 may selectably display a method of editing. Then, the editing unit 13 may edit an entrance image, by using a selected method.

Further, in shop entry person information stored by the storage unit 17, an executed method of editing may be registered in association with each shop visitor. Then, the editing unit 13 may edit, at a time of a next shop visit, an entrance image, by using a method different from a method executed in a past. Note that, based on a feature value of a face image of a shop visitor, information at a time of a past shop visit of each shop visitor can be extracted from among pieces of information registered in shop visitor information.

Other configurations of the shop system 10 are similar to those of the first and the second example embodiments.

According to the shop system 10 of the present example embodiment, an advantageous effect similar to that of the first and the second example embodiments is achieved. Further, according to the shop system 10 of the present example embodiment, an image is not edited by using a similar method for all shop visitors but an image can be edited by using a method according to a person attribute of a shop visitor. An image can be edited by using a method suitable for each shop visitor, and therefore a degree of satisfaction for a edited image is increased. Thereby, a shop visitor enjoys image capture work prior to shop entry. As a result, resistance to image capture work prior to shop entry can be further reduced.

### Modified Example

Herein, a modified example applicable to all example embodiments is described. As described according to the example embodiments described above, while image capture work is performed for shop entry, when, instead of an image itself acquired by capturing an image of a shop visitor, a edited image acquired by editing the image is displayed in a browsable manner, it is conceivable that a plurality of shop visitors having visited a shop in a group (a family, friends, or the like) desire to browse and enjoy edited images for the visitors. Regardless of this, when a configuration in which an image is captured and shop entry is made one by one is made, convenience is poor.

Therefore, a shop system 10 may include a means for receiving, from a shop visitor, input for specifying the number of shop visitors. Then, a processing unit 16 may set an entry control apparatus 20 into an entry-allowed state, according to a fact that entrance images for a specified number of persons are registered in shop entry person information.

When the entry control apparatus 20 is set, while shop visitors in a group perform image capture work, into an entry-allowed state every time an entrance image of each shop visitor is registered in shop entry person information, each shop visitor thinks that, according to a fact that his/her own image capture work is finished, the visitor should enter a shop when the entry control apparatus 20 is set into an entry-allowed state. Further, when the entry control apparatus 20 is set into an entry-allowed state at a needless timing, inconvenience in which an unrelated person enters a shop in the gap, or inconvenience in which, when the entry control apparatus 20 is needlessly operated, a processing load is increased or the like may occur. As in the modified example, in a case of a configuration in which the entry control apparatus 20 is set into an entry-allowed state according to a fact that entrance images for a specified number of persons are registered in shop entry person information, the inconvenience can be reduced.

Note that, in the present description, “acquisition” may include a matter that “a local apparatus fetches data stored in another apparatus or a storage medium (active acquisition)”, based on user input or based on an instruction from a program, for example, a matter that reception is executed by making a request or an inquiry to another apparatus. a matter that reading is executed by accessing another apparatus or a storage medium, and the like. Further, “acquisition” may include a matter that “data output from another apparatus are input to a local apparatus (passive acquisition)”, based on user input or based on an instruction from a program, for example, a matter that data distributed (or transmitted, reported on a push basis, or the like) are received, and the like. Furthermore, “acquisition” may include a matter that selective acquisition is executed from among received pieces of data or information, and a matter that “new data are generated by data editing (conversion into text, data rearrangement, partial data extraction, file-format modification, and the like), and the new data are acquired”.

The whole or part of the example embodiments described above can be described as, but not limited to, the following supplementary notes.
1. A shop system including:
   an acquisition unit that acquires an entrance image generated by a camera installed at a shop entrance;
   a detection unit that detects a face of a person in the entrance image;
   a editing unit that generates a edited image acquired by editing the entrance image in which a face of a person is detected;
   a display control unit that displays, on a display unit, the edited image;
   a determination unit that determines whether the entrance image prior to editing of the edited image being displayed by the display unit at a predetermined image capture timing satisfies a predetermined condition; and
   a processing unit that registers, when the predetermined condition is satisfied, the entrance image determined as satisfying the predetermined condition in shop entry person information stored by a storage unit, and also setting an entry control apparatus installed at a shop entrance into an entry-allowed state.
2. The shop system according to supplementary note 1, wherein
   the display unit displays the edited image in such a way as to be browsable by a person whose image is being captured by a camera installed at the shop entrance.
3. The shop system according to supplementary note 1 or 2, wherein
   the predetermined condition is that a predetermined feature value of a face of a person can be extracted.
4. The shop system according to any one of supplementary notes 1 to 3, wherein
   a feature value extracted from a face of a person included in the entrance image and a feature value extracted from a face of a person included in the edited image are not matched with each other.
5. The shop system according to any one of supplementary notes 1 to 4, wherein
   the processing unit executes processing of storing, in the storage unit, the edited image being displayed by the display unit at the predetermined image capture timing and providing the stored image for a shop visitor.
6. The shop system according to supplementary note 5, wherein
   the processing unit deletes, from the storage unit at a predetermined timing, the entrance image and the edited image stored in the storage unit, and
   a timing of deleting the edited image is earlier than a timing of deleting the entrance image.
7. The shop system according to any one of supplementary notes 1 to 6, wherein
   the editing unit estimates a person attribute, based on a face of a person detected in the entrance image, and edits the entrance image by using a method according to an estimation result.
8. A processing method including,
   by a computer:
      acquiring an entrance image generated by a camera installed at a shop entrance;
      detecting a face of a person in the entrance image;
      generating a edited image acquired by editing the entrance image in which a face of a person is detected;
      displaying, on a display unit, the edited image;
      determining whether the entrance image prior to editing of the edited image being displayed by the display unit at a predetermined image capture timing satisfies a predetermined condition; and
      registering, when the predetermined condition is satisfied, the entrance image determined as satisfying the predetermined condition in shop entry person information stored by a storage unit, and also setting an entry control apparatus installed at a shop entrance into an entry-allowed state.
9. A program causing a computer to function as:
   an acquisition unit that acquires an entrance image generated by a camera installed at a shop entrance;
   a detection unit that detects a face of a person in the entrance image;
   a editing unit for generating a edited image acquired by editing the entrance image in which a face of a person is detected;
   a display control unit that displays, on a display unit, the edited image;
   a determination unit that determines whether the entrance image prior to editing of the edited image being displayed by the display unit at a predetermined image capture timing satisfies a predetermined condition; and
   a processing unit that registers, when the predetermined condition is satisfied, the entrance image determined as satisfying the predetermined condition in shop entry person information stored by a storage unit, and also setting an entry control apparatus installed at a shop entrance into an entry-allowed state.

While the invention of the present application has been described with reference to example embodiments (and examples) thereof, the invention of the present application is not limited to these example embodiments (and examples). It will be understood by those of ordinary skill in the art that various changes in form and details may be made therein without departing from the spirit and scope of the invention of the present application as defined by the claims.

This application is based upon and claims the benefit of priority from Japanese patent application No. 2020-070797, filed on Apr. 10, 2020, the disclosure of which is incorporated herein in its entirety by reference.

### Reference Signs List

1A Processor
2A Memory
3A Input/output I/F
4A Peripheral circuit
5A Bus
10 Shop system
11 Acquisition unit
12 Detection unit
13 Editing unit
14 Display control unit
15 Determination unit
16 Processing unit
17 Storage unit
20 Entry control apparatus
30 Controller
40 Camera
50 Display apparatus

What is claimed is:

1. A shop system including:
at least one memory configured to store one or more instructions; and
at least one processor configured to execute the one or more instructions to:
acquire an entrance image generated by a camera installed at a shop entrance;
detect a face of a person in the entrance image;
generate a edited image acquired by editing the entrance image in which a face of a person is detected;
display, on display unit, the edited image;
determine whether the entrance image prior to editing of the edited image being displayed by the display unit at a predetermined image capture timing satisfies a predetermined condition; and
register, when the predetermined condition is satisfied, the entrance image determined as satisfying the predetermined condition in shop entry person information stored by storage unit, and also setting an entry control apparatus installed at a shop entrance into an entry-allowed state.

2. The shop system according to claim 1, wherein
the processor is further configured to execute the one or more instructions to display, on the display unit, the edited image in such a way as to be browsable by a person whose image is being captured by a camera installed at the shop entrance.

3. The shop system according to claim 1, wherein
the predetermined condition is that a predetermined feature value of a face of a person can be extracted.

4. The shop system according to claim 1, wherein
a feature value extracted from a face of a person included in the entrance image and a feature value extracted from a face of a person included in the edited image are not matched with each other.

5. The shop system according to claim 1, wherein
the processor is further configured to execute the one or more instructions to execute processing of storing, in the storage unit, the edited image being displayed by the display unit at the predetermined image capture timing and providing the stored image for a shop visitor.

6. The shop system according to claim 5, wherein
the processor is further configured to execute the one or more instructions to delete, from the storage unit at a predetermined timing, the entrance image and the edited image stored in the storage unit, and
a timing of deleting the edited image is earlier than a timing of deleting the entrance image.

7. The shop system according to claim 1, wherein
the processor is further configured to execute the one or more instructions to estimate a person attribute, based on a face of a person detected in the entrance image, and edits the entrance image by using a method according to an estimation result.

8. A processing method including,
by a computer:
acquiring an entrance image generated by a camera installed at a shop entrance;
detecting a face of a person in the entrance image;
generating a edited image acquired by editing the entrance image in which a face of a person is detected;
displaying, on display unit, the edited image;
determining whether the entrance image prior to editing of the edited image being displayed by the display unit at a predetermined image capture timing satisfies a predetermined condition; and
registering, when the predetermined condition is satisfied, the entrance image determined as satisfying the predetermined condition in shop entry person information stored by storage unit, and also setting an entry control apparatus installed at a shop entrance into an entry-allowed state.

9. A non-transitory storage medium storing a program causing a computer to:
acquire an entrance image generated by a camera installed at a shop entrance;
detect a face of a person in the entrance image;
generate a edited image acquired by editing the entrance image in which a face of a person is detected;
display, on display unit, the edited image;
determine whether the entrance image prior to editing of the edited image being displayed by the display unit at a predetermined image capture timing satisfies a predetermined condition; and
register, when the predetermined condition is satisfied, the entrance image determined as satisfying the predetermined condition in shop entry person information stored by storage unit, and also setting an entry control apparatus installed at a shop entrance into an entry-allowed state.
